(19) United States
(12) Patent Application Publication
(10) Pub. No.: US 2024/0108584 A1
(43) Pub. Date: Apr. 4, 2024

(54) POLYMERIC MICROPARTICLES, COMPOSITIONS, AND METHODS FOR SUSTAINED RELEASE OF AN ACTIVE AGENT SUSCEPTIBLE TO ABUSE

(71) Applicant: Ohio State Innovation Foundation, Columbus, OH (US)

(72) Inventors: John LANNUTTI, Grove City, OH (US); Fan FAN, Xi'an, Shaanxi Province (CN); Andrea TEDESCHI, Columbus, OH (US)

(21) Appl. No.: 18/274,964

(22) PCT Filed: Jan. 31, 2022
(86) PCT No.: PCT/US2022/014589

§ 371 Date: Jul. 28, 2023

Related U.S. Application Data
(60) Provisional application No. 63/143,554, filed on Jan. 29, 2021.

(51) Int. Cl. A61K 9/50; A61K 31/197; A61K 47/10; A61K 47/34; A61P 25/28

(52) U.S. Cl. CPC A61K 9/5031; A61K 31/197; A61K 47/10; A61K 47/34; A61P 25/28

(57) ABSTRACT

Described are polymeric microparticles, compositions and method of making and using. The polymeric microparticles can include a polymeric core comprising a first polymer and an active agent susceptible to abuse; and a polymeric shell comprising a second polymer.

### CROSS-REFERENCE TO RELATED APPLICATIONS

This application claims benefit of U.S. Provisional Application No. 63/143,554, filed Jan. 29, 2021, which is hereby incorporated herein by reference in its entirety.

### STATEMENT REGARDING FEDERALLY SPONSORED RESEARCH OR DEVELOPMENT

This invention was made with government support under grant/contract number L15AC00146 awarded by the Bureau of Land Management. The government has certain rights in the invention.

### BACKGROUND

Traumatic brain and spinal cord injury cause devastating neurological deficits and long-term disability associated with chronic pain syndromes, spasticity and muscle paralysis due to detrimental structural and functional alteration in neuronal circuits. Currently, the cellular and molecular mechanisms that cause or contribute to pathophysiological changes in central nervous system structure and function are not well controlled. A number of studies, including ours, have demonstrated a remarkable convergence between structural and functional organization of neuronal circuits and expression of α2δ subunits of voltage gated calcium channels (VGCC). α2δ subunits positively regulate synaptic transmission by increasing plasma membrane expression of VGCC. However, these subunits may also play a pathological role following axonal injury. Expression of α2δ1 and α2δ2 increases following axonal injury, resulting in aberrant neuron activities associated with chronic pain and post-traumatic epilepsy.

There is a need to develop more effective clinical interventions aimed to improve neurological function and quality of life in individuals afflicted by brain and spinal cord injury and reduce the impact of neurodegenerative diseases, which are a huge economic and emotional burden on society.

The compositions and methods disclosed herein address these and other needs.

### SUMMARY

Provided herein are polymeric microparticles, compositions, and methods using and making. The polymeric microparticles can include a polymeric core and a polymeric shell. In some embodiments, the polymeric core can include a first polymer and an active agent susceptible to abuse. In some embodiments, the polymeric shell can include a second polymer. In some embodiments, the polymeric shell can further include a dispersing agent. In some embodiments, the polymeric microparticles can be injectable. In some embodiments, the active agent susceptible to abuse can be a gabapentinoid, or a pharmaceutically acceptable salt thereof. In some embodiments, the gabapentinoid can be gabapentin or pregabalin, or a pharmaceutically acceptable salt thereof. In some embodiments, the active agent susceptible to abuse can be present in a weight loading of from 0.1 wt. % to 50 wt. % in the polymeric microparticle.

In some embodiments, the first and second polymers can be biocompatible polymers. In some embodiments, at least one of the first or second polymers can be a non-erodible biocompatible polymer. In some embodiments, the dispersing agent can include polymers such as polyethylene glycol, poloxamers, or a combination thereof.

In some embodiments, the dispersing agent can be present in an amount of from 0.01 wt. % to 10 wt. %. In some embodiments, the polymeric microparticles can have an average diameter ranging from 0.1 microns to 100 microns. In some embodiments, the microparticles exhibits sustained, zero-ordered release the active agent susceptible to abuse over a period of days to weeks. In some embodiments, the microparticles release the drug over a period of days to weeks.

Provided herein are also pharmaceutical compositions for localized drug delivery. In some embodiments, the composition comprising polymeric microparticles described herein is dispersed within a pharmaceutically acceptable carrier. In some embodiments, the pharmaceutically acceptable carrier can include a dispersing agent as described herein.

Provided herein are also methods of preparing the polymeric microparticles described herein comprising (a) dissolving or dispersing the first polymer in an organic solvent to generate a first polymer solution/dispersion; (b) dissolving or dispersing the second polymer in an organic solvent to generate a shell solution; (c) adding the active agent susceptible to abuse to the first polymer solution/dispersion of step (a) to generate a core solution; (d) electrospraying the core solution and the shell solution onto a pre-treated dish; and (e) collecting the polymeric microparticles on the pre-treated dish. In some embodiments, the method can further include adding a dispersing agent to the shell solution of step (b).

In some embodiments, the method of making the polymeric microparticles described herein uses coaxial electrospraying including dissolving a first polymer and an active agent susceptible to abuse in a first solvent to form a core solution; dissolving a second polymer in a second solvent to form a shell solution; flowing the core solution through an inner coaxial needle and the shell solution through an outer coaxial needle concurrently under an electric field; and collecting the resulting microparticles. In some embodiments, the method can further include adding a dispersing agent as described herein to the shell solution.

In some embodiments, the first solvent and second solvent are the same solvent. In some embodiments, the first solvent and second solvent comprise dichloromethane, tetrahydrofuran, 1,1,1,3,3,3-hexafluoro-2-propanol, dimethylformamide, or any combination thereof. In some embodiments, the first and second polymers are biocompatible polymers. In some embodiments, at least one of the first or second polymers can be a non-erodible biocompatible polymer. In some embodiments, the biocompatible polymer can be present in an amount ranging from 1 wt. % to 3 wt. % of the core solution, shell solution or both. In some embodiments, the non-erodible biocompatible polymer can be present in an amount ranging from 1 wt. % to 3 wt. % of the core solution, shell solution or both.

Also described are methods for treating a neurodegenerative disorder in a subject in need thereof, including administering an effective amount of the polymeric microparticles or the pharmaceutical composition described herein.

In some embodiments, neurodegenerative disorder can be the result of a brain, spinal cord or optic nerve injury. In some embodiments, the neurodegenerative disorder can be Alzheimer's disease, Parkinson's disease, prion disease, motor neuron disease, Huntington's disease, spinocerebellar ataxia, spinal muscular atrophy, or any combination thereof.

The details of one or more embodiments of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 shows a schematic of the core-shell electrospraying process with an inset showing the desired oxygen sensing microparticle.

FIGS. 2A-2C show SEM images of PSU-PSU core-shell particles using DCM at different core-shell flow rate ratios (2A) 0.1/0.5 mL/hr (2B) 0.3/0.5 mL/hr (2C) 0.5/0.5 mL/hr. Source-to-collector distance: 20 cm. Applied voltage: 20 kV. Magnification of main images: 20,000×. Magnification of inset images: 100,000×.

FIGS. 3A-3C show SEM images of PSU-PSU core-shell particles using 75:25 DCM-HFP blend at different core-shell flow rate ratios (3A) 0.1/0.5 mL/hr (10,000×) (3B) 0.3/0.5 mL/hr (20,000×) (3C) 0.5/0.5 mL/hr (20,000×). Source-to-collector distance: 20 cm. Applied voltage: 20 kV. Particles electrosprayed into PBS.

FIGS. 4A-4C show core solution: 1 wt % PSU in 75/25 DCM/HFP+0.5 wt % PdTFPP, 0.3 mL/hr and shell solution: 1 wt % PSU in 75/25 DCM/HFP+1 wt % Pluronic F-127, 0.5 mL/hr. (4A) Combined fluorescent/DIC mode TIRF images of electrosprayed particles. (4B-4C) TIRF images of these particles in distilled water with dissolved oxygen contents of (4B) 0.21 mg/L and (4C) 8.7 mg/L.

FIGS. 5A-5F show SEM images of PSU-PSU core-shell particles using DMF electrosprayed in accordance with variable applied voltage: (5A) 14 kV (5B) 15 kV (5C) 16 kV (5D) 17 kV (5E) 18 kV (5F) 19 kV. Source-to-collector distance: 15 cm. Core flow rate: 0.3 mL/hr. Shell flow rate: 1 mL/hr. Magnification: 10,000×.

FIGS. 6A-6B show fluorescent mode TIRF images of these particles in distilled water with dissolved oxygen contents of (6A) 0.13 mg/L and (6B) 8.1 mg/L. Core solution: 1 wt % PSU in THF+1 wt % PdTFPP, 0.3 mL/hr and shell solution: 1 wt % PSU in THF+1 wt % Pluronic F-127, 1 mL/hr. A-B).

FIGS. 7A-7E show optical images (Nikon Eclipse LV150, Melville, NY) of PSU-PSU particle dispersion in PBS (1×). (7A) Particles in PBS. (7B) Sample (7A) after 5 minutes of bath sonication. (7C) Sample (7B) after adding Pluronic F-127 into PBS (1×) (F-127 concentration is 1 mg/mL). (7D) Particle suspension from (9C) after 5 minutes of bath sonication. (7E) Optical microscope image of particle suspension from (7D) four days after sonication. Post-sonication dispersion stability appeared to be good; no signs of reagglomeration were visible.

FIGS. 8A-8C show optical microscope (Zeiss Axio Observer Z1, Oberkochen, Germany) images of particle injection. (8A) Empty glass-pulled micropipette. (8B) PSU-PSU particle suspension flowing in the same micropipette. (8C) Micropipette from (8B) at a larger scale.

FIG. 9 shows a schematic of the core shell microparticle.

FIGS. 10A-10B show images of electrosprayed particles using core. 1 wt % PSU in DCM/IHFP, 0.1 mL/hr and shell: PSU in DCM/HFP+1 wt % Pluronic F-127, 0.5 mL/hr. The DCM/HFP ratio was (10A) 50/50 and (10B) 65/35. Source-to-collector distance. 20 cm. Applied voltage: 20 kV Particles electrosprayed into PBS.

FIG. 11 shows SEM image of PSU-PSU core-shell particles using THF electrosprayed at 14 kV Source-to-collector distance: 15 cm. Core flow rate: 0.3 mL/hr. Shell flow rate: 1 mL/hr.

FIGS. 12A-12G show (12A) schematic of the thoracic (T11) spinal cord injury (SCI) in adult mice. GM: gray matter, WM: white matter. (12B) Timeline of the experimental paradigm. (12C) Three-dimensional scan of the unsectioned mouse spinal cord (R: rostral, C: caudal, MIP: max intensity projection). Coumarin 6 loaded microparticles are clearly visible at the lesion site. The use of higher viscosity polyethylene glycol focalized the injected particle loads at the wound site. Fibronectin stains the fibrotic core of the lesion. Glial fibrillary acidic protein (GFAP) stains the astrocytic limitants of the injury. Yellow asterisk indicates the lesion epicenter. Scar bar: 100 microns. (12D) Three-dimensional rendering of the injured spinal cord containing coumarin 6 microparticles. (12E) Representative frames of skilled walking 28 days after thoracic (T11) SCI in mice. The black arrow indicates a footfall in a mouse injected with control (CTR) microparticles. (12F) Behavioral recovery in SCI mice was assessed using the skilled walking test and (12G) Von Frey test.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s) but may also include other elements or steps not expressly recited. Furthermore, as used herein, the use of the terms “a”, “an”, and “the” when used in conjunction with an element may mean “one,” but it is also consistent with the meaning of“one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic. It is understood that this is typically in relation to some standard or expected value, in other words it is relative, but that it is not always necessary for the standard or relative value to be referred to.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

Polymeric Microparticles

Described herein are injectable polymeric microparticles including a polymeric core and a polymeric shell. In some embodiments, the polymeric core can include a first polymer and an active agent susceptible to abuse. In some embodiments, the polymeric shell can include a second polymer. In some embodiments, the polymeric shell can further include a dispersing agent.

Active agents susceptible to abuse can be drugs or salts thereof that have a potential to be abused or which are susceptible to abuse. Suitable active agents susceptible to abuse include can include, but are not limited to, those commonly prescribed for relieving pain such as barbiturates and opioids. A few drug compounds for pain relief include, but are not limited to, codeine, phenazocine, tilidine, tramadol, meperidine, sufentanil, prodine, methadone, pentazocine, oxycodone, oxymorphone, hydrocodone, hydromorphone, tapentadol, morphine, buprenorphine, and fentanyl. Other drugs that can be misused for non-therapeutic purposes have hallucinogenic properties or otherwise affect the central nervous system, including stimulants such as amphetamines.

Some other drugs that can be the subject of abuse include, but are not limited to, alfentanil; allobarbital; allylprodine; alphaprodine; alprazolam; amfepramone; amphetamine; amphetaminil; amobarbital; anileridine; atomoxetine; apocodeine; barbital; benzodiazepine, benzylmorphine; bezitramide; bromazepam; brotizolam; buprenorphine butobarbital; butorphanol; buspirone; camazepam; carisoprodol, chlorodiazepoxide; clobazam; clonazepam; clonitazene; clorazepate; clotiazepam; cloxazolam; cocaine; codeine, cyclobarbital; cyclorphan; cyprenorphine; delorazepam; desomorphine; dextroamphetamine, dexmethylphenidate, dextromoramide; dextropropoxyphen; dezocine; diampromide; diamorphone; diazepam; dihydrocodeine; dihydromorphine; dimenoxadol; dimepheptanol; dimethylthiambutene; dioxaphetyl butyrate; dipipanone; dronabinol; eptazocine; ephedrine, estazolam; eszopiclone, ethoheptazine; ethylmethylthiambutene; ethyl loflazepate; ethylmorphine; etonitazene; etorphine; fencamfamine; fenethylline; fenproporex; fentanyl, fludiazepam; flunitrazepam; flurazepam; guanfacine; gabapentin; halazepam; haloxazolam; heroin; hydrocodone, hydromorphone, hydroxypethidine; hydroxymethyl morphinane; isomethadone; ketazolam; ketobemidone; levomethadyl acetate; levomethadone; levorphanol; levophenacylmorphane; lofentanil; loprazolam; lorazepam; lormetazepam; lisdexamfetamine; mazindol; medazepam; mefenorex; meprobamate; meptazinol; metazocine; methadone, methylmorphine; methamphetamine; methaqualone; methylphenidate; methylphenobarbital; methyprylon; meperidine, metopon; midazolam; modafinil; morphine, myrophine; nabilone; nalbuphine; nalorphine; narceine; nicomorphine; nimetazepam; nitrazepam; nordazepam; norlevorphanol; normethadone; normorphine; norpipanone; opium; oxazepam; oxazolam; oxycodone, oxymorphone, pernoline; pentazocine, pentobarbital; pethidine; phenadoxone; phenomorphan; phenoperidine; piminodine; pholcodine; phenmetrazine; phenobarbital; phentermine; phenazocine, pinazepam; pipradrol; piritramide; prazepam; pregabalin; prodine, profadol; proheptazine; promedol; properidine; propoxyphene; pseudoephedrine, remifentanil; secbutabarbital; secobarbital; serdexmethylphenidate; sufentanil, tapentadol, temazepam; tetrazepam; tilidine; tramadol; triazolam; vinylbital; zolpidem, or any combination thereof. The drugs include any pharmacologically active stereoisomeric compounds, as well as derivatives of the base drug such as esters and salts, including any solvates thereof. The active agent susceptible to abuse can be present in the composition in an amount effective for the intended therapeutic purpose. These amounts are well known in the art. All of the active agents embraced by the present disclosure are known per se, as are the doses at which they can be given safely and effectively for the intended therapeutic purpose.

In some embodiments, the active agent susceptible to abuse can be a gabapentinoid, or a pharmaceutically acceptable salt thereof. In some embodiments, the gabapentinoid can be gabapentin or pregabalin, or a pharmaceutically acceptable salt thereof.

In some embodiments, the gabapentinoid may be present in a weight loading of from 0.1 wt. % to 50 wt. % in the polymeric microparticle. For example, the gabapentinoid may be present in a weight loading of from 1 wt. % to 20 wt. %, 3 wt. % to 15 wt. %, or 5 wt. % to 15 wt. % in the polymeric microparticle. In some embodiments, the gabapentinoid may be present in a weight loading of from 5.8 wt. % to 13.3 wt. % in the polymeric microparticle.

In some embodiments, the first and second polymers are biocompatible polymers. In some embodiments, the first and/or second polymer are a non-erodible biocompatible polymer. In some embodiments, the first and second polymer are a non-erodible biocompatible polymer. In some embodiments, the first polymer is a non-erodible biocompatible polymer and the second polymer is an erodible biocompatible polymer. In some embodiments, the first polymer is an erodible biocompatible polymer and the second polymer is a non-erodible biocompatible polymer. A biocompatible polymer refers to polymers which do not have toxic or injurious effects on biological functions. Biocompatible polymers include natural or synthetic materials. Examples of biocompatible polymers include, but are not limited to, collagen, poly (alpha esters such as poly (lactate acid), poly (glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polycaprolactone, polytetrafluoroethylene, polythioether, polytriazole, polyurethane, polyvinyl, polyvinylidene fluoride, or copolymers and blends thereof.

In some embodiments, the biocompatible polymer comprises a polysulfone, polycaprolactone, or any combination thereof. In some embodiments, the biocompatible polymer comprises a polysulfone. In some embodiments, the non-erodible biocompatible polymer can be polysulfone, polyethersulfone, nylon, polyethylene, polypropylene, or polyvinylchloride.

In some embodiments, the dispersing agent comprises polymers such as polyethylene glycol, poloxamers, or a combination thereof. In some embodiments, a poloxamer can be a polyoxyethylene-polyoxypropylene block copolymer defined by (PEO)ₓ(PPO)_{y}(PEO)ₓ, wherein PEO is poly(ethylene oxide), PPO is poly(propylene oxide), x can each be an integer from 2 to 130, and y can be an interfere from 15 to 67. In some embodiments, the poloxamer can be (PEO)₂₀(PPO)₇₀(PEO)₂₀. (PEO)₃₈(PPO)₂₉(PEO)₃₈, (PEO)₁₃₆(PPO)₅₂(PEO)₁₃₆, (PEO)₈₂(PPO)₃₁(PEO)₈₂, (PEO)₉₅(PPO)₆₂(PEO)₉₅, (PEO)₅(PPO)₆₈s(PEO)₅, or (PEO)₁₀₁(PPO)₅₆(PEO)₁₀₁. In some embodiments, the poloxamer is poloxamer 407. In some embodiments, the poloxamer is poloxamer 188. Other examples include Pluronic F68, Pluronic F108, Pluronic P123 or Pluronic L121. In some embodiments, the dispersing agent can be present in an amount of from 0 to 10 wt. %. For example, the dispersing agent can be present in an amount from 0.1 wt. % to 1 wt. %, from 0.5 wt. % to 1 wt. %, from 0.5 wt. % to 5 wt. %, or from 1 wt. % to 10 wt. % in the polymeric microparticle. In some embodiments, the dispersing agent can be present in an amount of from 0.60 to 0.65 wt. % in the polymeric microparticle.

In some embodiments, the polymeric microparticles can have an average diameter ranging from 0.1 microns to 100 microns. For example, the polymeric microparticles can have an average diameter of from 0.90 microns to 1.50 microns, from 1.06 microns to 1.17 microns, from 1.0 microns to 1.5 microns, from 1.0 microns to 1.2 microns, from 0.92 microns to 1.20 microns, from 0.1 microns to 10 microns, from 0.5 microns to 2 microns, from 0.5 microns to 25 microns, from 0.5 microns to 50 microns, from 0.5 microns to 75 microns, or from 1 micron to 10 microns. In some embodiments, the polymeric microparticles can have an average diameter of about 1.06±0.14 microns. In some embodiments, the polymeric microparticles can have an average diameter of about 1.17±0.17 microns. In some embodiments, the microparticles release the drug over a period of days to weeks.

In some embodiments, the polymeric microparticles exhibit sustained, zero-order release. In some embodiments, the polymeric microparticles exhibit sustained, zero-order release for at least 30 minutes, at least 1 hour, at least 2 hours, at least 3 hours, at least 6 hours, at least 12 hours, at least 24 hours, at least 48 hours, at least 72 hours, at least 7 days, at least 14 days, or at least 21 days. In some embodiments, the polymeric microparticles exhibit sustained, zero-order release for 30 days or less, 21 days or less, 14 days or less, 7 days or less, 72 hours or less, 48 hours or less, 24 hours or less, 12 hours or less, 6 hours or less, 3 hours or less, 2 hours or less, or 1 hour or less. The polymeric microparticles exhibit sustained, zero-order release for a period of time ranging from any of the minimum values described above to any of the maximum values described above.

Pharmaceutical Compositions

Provided herein are also pharmaceutical composition including a population of polymeric microparticles described herein dispersed within a pharmaceutically acceptable carrier. In some embodiments, the composition can include multiple populations of polymeric microparticles each population of polymeric microparticle including a specific sustained release profile.

For example, when administered to a subject, the disclosed compositions can release multiple populations of polymeric microparticles at certain periods of time, rather than all at once. For example, the disclosed compositions can include a first population of polymeric microparticles releasing the active agent over a period of 24 hours beginning immediately. In some embodiments, the compositions can include a second population of polymeric microparticles releasing the active agent within 24 hours of administration releasing the active agent over a period of 48 hours. In some embodiments, an additional population of polymeric microparticles then releases the active agent within 48 of administration over a period of 672 hours.

In some embodiments, for example, the compositions can include a first population of polymeric microparticles releasing the active agent susceptible to abuse immediately or within about 60 minutes of administration for a period of 24 hours beginning immediately; a second population of polymeric microparticles releases the active agent susceptible to abuse 24 hours after the initial administration for a period of 24 hours. In some embodiments, for example, the compositions can include a third population of polymeric microparticles releasing the active agent susceptible to abuse immediately within 48 hours of administration over a period of 24 hours.

In some embodiments, the pharmaceutical composition exhibits sustained, zero-order release. In some embodiments, the polymeric microparticles exhibit sustained, zero-order release for at least 14 days. In some embodiments, the polymeric microparticles exhibit sustained, zero-order release for 6 hours or less.

In some embodiments, the pharmaceutically acceptable carrier can include a dispersing agent. In some embodiments, the dispersing agent can include a polymer such as polyethylene glycol, poloxamers, or a combination thereof. In some embodiments, a poloxamer can be a polyoxyethylene-polyoxypropylene block copolymer defined by (PEO)ₓ-(PPO)_{y}-(PEO)ₓ, wherein PEO is poly(ethylene oxide), PPO is poly(propylene oxide), x can each be an integer from 2 to 130, and y can be an interfere from 15 to 67. In some embodiments, the poloxamer can be (PEO)₂₀(PPO)₇₀(PEO)₂₀, (PEO)₃₈(PPO)₂₉(PEO)₃₈, (PEO)₁₃₆(PPO)₅₂(PEO)₁₃₆, (PEO)₈₂(PPO)₃₁(PEO)₈₂, (PEO)₉₅(PPO)₆₂(PEO)₉₅, (PEO)₅(PPO)₆₈(PEO)₅, or (PEO)₁₀₁(PPO)₅₆(PEO)₁₀₁. In some embodiments, the poloxamer is poloxamer 407. In some embodiments, the poloxamer is poloxamer 188. Other examples include Pluronic F68, Pluronic F108, Pluronic P123 or Pluronic L121. In some embodiments, the pharmaceutically acceptable carrier can include polyethylene glycol. In some embodiments, the pharmaceutical composition is an injectable pharmaceutical composition. The use of higher viscosity polyethylene glycol helps focalize injected particle loads at the wound site.

Methods of Making

Provided herein are also methods of preparing the polymeric microparticles described herein comprising (a) dissolving or dispersing the first polymer in an organic solvent to generate a first polymer solution/dispersion; (b) dissolving or dispersing the second polymer in an organic solvent to generate a shell solution; (c) adding the active agent susceptible to abuse to the first polymer solution/dispersion of step (a) to generate a core solution; (d) electrospraying the core solution and the shell solution onto a pre-treated dish; and (e) collecting the polymeric microparticles on the pre-treated dish.

In some embodiments, the method can further include adding a dispersing agent to the shell solution of step (b). In some embodiments, the shell solution has a flow rate of less than the flow rate of the core solution.

In some embodiments, the method of making the polymeric microparticles described herein uses coaxial electrospraying including dissolving a first polymer and an active agent susceptible to abuse in a first solvent to form a core solution; dissolving a second polymer in a second solvent to form a shell solution; flowing the core solution through an inner coaxial needle and the shell solution through an outer coaxial needle concurrently under an electric field; and collecting the resulting microparticles. In some embodiments, the method can further include adding a dispersing agent to the shell solution.

In some embodiments, the first solvent and second solvent are the same solvent. In some embodiments, the first solvent and second solvent comprise dichloromethane, tetrahydrofuran, 1,1,1,3,3,3-hexafluoro-2-propanol, dimethylformamide, or any combination thereof.

In some embodiments, the first and second polymers are biocompatible polymers. In some embodiments, the first and/or second polymer are a non-erodible biocompatible polymer. In some embodiments, the first and second polymer are a non-erodible biocompatible polymer. In some embodiments, the first polymer is a non-erodible biocompatible polymer and the second polymer is an erodible biocompatible polymer. In some embodiments, the first polymer is an erodible biocompatible polymer and the second polymer is a non-erodible biocompatible polymer. A biocompatible polymer refers to polymers which do not have toxic or injurious effects on biological functions. Biocompatible polymers include natural or synthetic materials. Examples of biocompatible polymers include, but are not limited to, collagen, poly (alpha esters such as poly (lactate acid), poly (glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polycaprolactone, polytetrafluoroethylene, polythioether, polytriazole, polyurethane, polyvinyl, polyvinylidene fluoride, or copolymers and blends thereof. In some embodiments, the biocompatible polymer comprises a polysulfone, polycaprolactone, or any combination thereof. In some embodiments, the biocompatible polymer comprises a polysulfone.

The term “non-erodible biocompatible polymer” refers to a biocompatible polymer that are water insoluble. In some embodiments, the non-erodible biocompatible polymer can be polysulfone, poly(ethylene-co-vinyl acetate), and (EVA), polyvinylalcohol, polyethersulfone or a nylon.

In some embodiments, the biocompatible polymer can be present in an amount of from 1 wt. % to 3 wt. % of the core solution, shell solution, or both, such as from 1 wt. % to 2 wt. %, from 1 wt % to 3 wt. %, or from 2 wt % to 3 wt % of the core solution, shell solution, or both.

In some embodiments, the polysulfone can be present in an amount of from 1 wt. % to 3 wt. % of the core solution, shell solution, or both, such as from 1 wt. % to 2 wt. %, from 1 wt % to 3 wt. %, or from 2 wt % to 3 wt % of the core solution, shell solution, or both.

Methods of Use

Described are polymeric microparticles or pharmaceutical compositions that can be used for localized drug delivery including administering to a subject in need thereof a therapeutically effective amount of the polymeric microparticles or the pharmaceutical composition.

Described are also method for sustained drug release including administering to a subject in need thereof a therapeutically effective amount of polymeric microparticles or a pharmaceutical composition described herein.

Described are also methods for treating, a neurodegenerative disorder in a subject in need thereof, including administering a therapeutically effective amount of the polymeric microparticles or the pharmaceutical composition described herein.

In some embodiments, neurodegenerative disorder is the result of a brain, spinal cord, optic nerve injury, or any combination thereof. In some embodiments, the neurodegenerative disorder is Alzheimer's disease, Parkinson's disease, prion disease, motor neuron disease, Huntington's disease, spinocerebellar ataxia, spinal muscular atrophy, or any combination thereof.

Methods of Administration

The microparticles as used in the methods described herein can be administered by any suitable method and technique presently or prospectively known to those skilled in the art. For example, the active components described herein can be formulated in a physiologically- or pharmaceutically acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active agent susceptible to abuse of their compositions can be a single administration, or at continuous and distinct intervals as can be readily determined by a person skilled in the art.

The compositions, as described herein, comprising an active agent susceptible to abuse and an excipient of some sort may be useful in a variety of medical and non-medical applications.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active agent susceptible to abuse may be administered in such amounts, time, and route deemed necessary in order to achieve the desired result. The exact amount of the active agent susceptible to abuse will vary from subject to subject, depending on the species, age, and general condition of the subject, the severity of the infection, the particular active ingredient, its mode of administration, its mode of activity, and the like. The active agent susceptible to abuse, whether the active agent susceptible to abuse itself, or the active agent susceptible to abuse in combination with an agent, is preferably formulated in dosage unit form for ease of administration and uniformity of dosage. It will be understood, however, that the total daily usage of the active agent susceptible to abuse will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active agent susceptible to abuse employed; and like factors well known in the medical arts.

The active agent susceptible to abuse may be administered by any route. In some embodiments, the active agent susceptible to abuse is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active agent susceptible to abuse (e.g., its stability in the environment of the gastrointestinal tract), the condition of the subject (e.g., whether the subject is able to tolerate oral administration), etc. The exact amount of an active agent susceptible to abuse required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agent susceptible to abuse and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the compositions as used in the methods described herein may be administered in combination or alternation with one or more additional active agents. Representative examples additional active agents include antipsychotic agents, anticonvulsant agents, analgesic, and cognition enhancing agents.

Representative examples of antipsychotic agents include, but are not limited to, acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine, zuclopenthixol, amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone, and zotepine.

Representative examples of anticonvulsant agents include, but are not limited to, acetazolamide, brivaracetam, carbamazepine, cenobamate, clobazam, clonazepam, diazepam, divalproex sodium, eslicarbazepine, ethosuximide, ethotoin, everolimus, felbamate, fosphenytoin, gabapentin, lacosamide, lamotrigine, levetiracetam, mephenytoin, metharbital, methazolamide, methsuximide, oxcarbazepine, phenobarbital, phensuximide, phenytoin, piracetam, pregabalin, primidone, rufinamide, sodium valproate, stiripentol, tiagabine, topiramate, trimethadione, valproic acid, vigabatrin, and zonisamide.

Representative examples of cognition enhancing agents include, but are not limited to, memantine, rivastigmine, galantamine, and donepezil.

Representative examples of analgesics include, but are not limited to, acetaminophen, aspirin, non-steroidal anti-inflammatory drugs, ibuprofen, naproxen, diclofenac, celecoxib, and paracetamol.

Additional factors could include anti-inflammatory compounds, trophic factors and specific receptor blockers important in the healing of a variety of biological insults.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

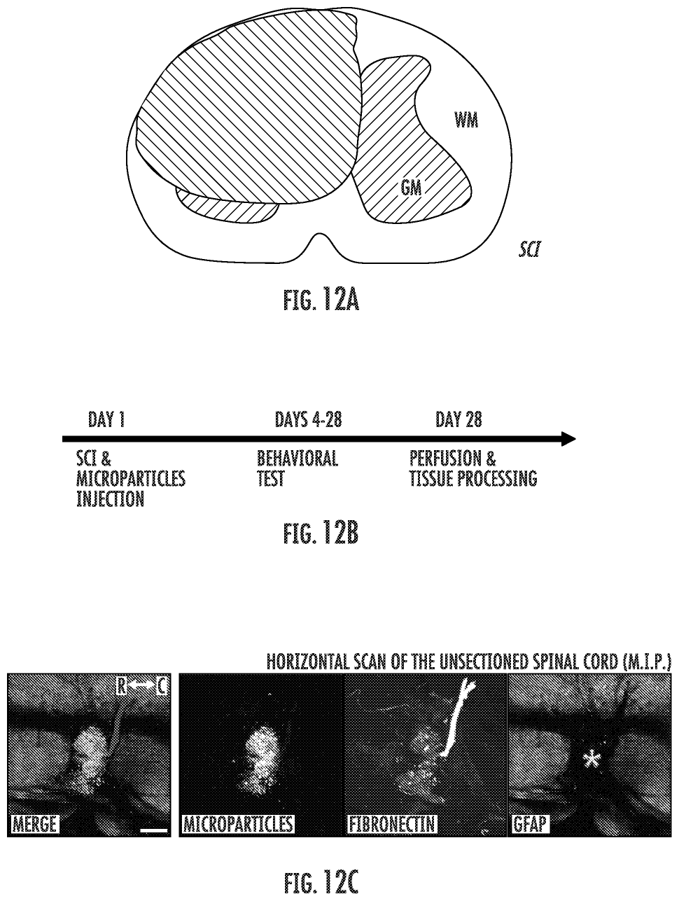

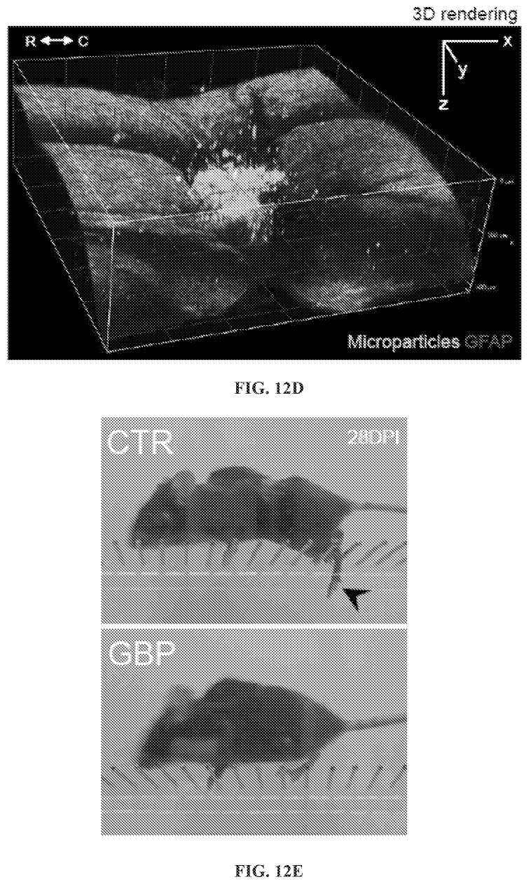

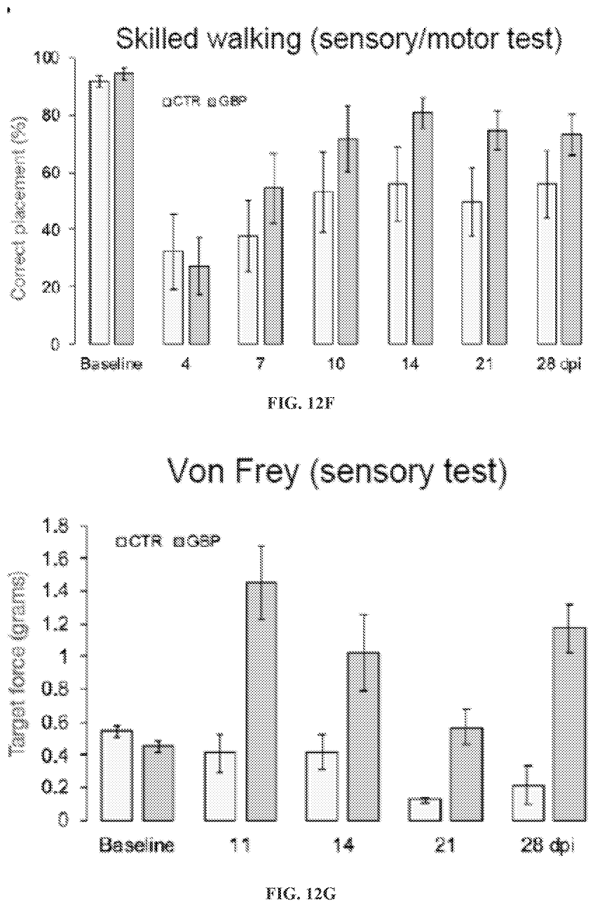

A ‘Smart’ Drug Delivery System (SDDS) was developed in vivo that counteracts maladaptive plasticity and neurodegeneration likely by pharmacologically blocking α2δ1/2 in the mouse spinal cord. The SDDS is made up of polymer-based, electrosprayed injectable polysulfone-polysulfone core-shell microspheres that encapsulate FDA-approved gabapentin to enable localized drug delivery. These electrosprayed particles have a small size and narrow size distribution, 1.06±0.14 μm, providing consistent release kinetics. The addition of the polymeric shell also improves the drug release kinetics by avoiding burst release. By establishing the means to disperse these microspheres efficiently in polyethylene glycol (PEG), a readily injectable, gravitationally stable dispersion has been demonstrated and applied to spinal cord injuries in adult mice (see FIGS. 12A-12G). PEG as a carrier has distinct advantages over aqueous suspensions as it does not ‘leach’ gabapentin from the microparticles. Due to its viscosity, PEG helps focalize injected particle loads at the site of injury.

### Example 1

Traumatic spinal cord injury (SCI) is a life-changing event with an extremely poor prognosis. This injury often results in physiological impairment and multisystem malfunction including disabilities, intractable neuropathic pain, and a range of extensive potential complications. Annually, approximately 10,000 Americans have a traumatic spinal cord injury (SCI). For many, the most visible aspect of this disability is either an inability to walk or to walk only using a slow, painful gait. To date, no effective treatments for SCI are available because of the complex pathophysiologic processes and the joint actions of multiple mechanisms triggered following the injury.

FIG. 12A provides a schematic rendering of an example of an injury (dark grey area) comprising a spinal cord deficit. Both white matter and grey matter—normal components of the spinal cord—are labeled as well. The timeline (FIG. 12B) shows that the injection of the gabapentin-releasing particles occurs shortly after the SCI itself. Behavioral testing occurs at points over the next month, followed by histology to examine the distribution of the injected particles as well as axonal morphology in the tissue. FIG. 12C shows how the particles were distributed within the wound site following injection. Coumarin 6 dye was added to the particles during the electrospraying process in exactly the same way as we add gabapentin to these same particles. This accounts for their vivid green color in these scans. GFAP (purple stain in FIGS. 12C and 12D) denotes the reactive astrocytes contained within the surrounding spinal cord, suggesting that gabapentin release can be highly targeted to the site of injury.

FIG. 12E shows the favorable recovery of mice treated with the gabapentin-releasing microparticles versus treatment with control (CTR) PSU particles that were electrosprayed without any gabapentin. The frames shown indicate that mice benefiting from these gabapentin-releasing particle injections were more sure-footed in skilled walking post-injury than mice having only the control particle injections. FIG. 12F shows the same result but more quantitatively. This result suggests that such treatments in humans might result in similar beneficial effects on recovery of neurological functions from SCI.

Finally, FIG. 12G shows that tactile sensory testing (also known as Von Frey) indicates that mice receiving the gabapentin-loaded particles show normalization of tactile sensitivity from early time points (e.g., 11d). This early recovery suggests that humans treated with these gabapentin-bearing particles might experience less of the long-term intractable neuropathic pain that often limits patient mobility and decreases quality of life.

### Example 2

Traumatic brain, spinal cord and optic nerve injury cause devastating neurological deficits and long-term disability due to detrimental structural and functional alteration in neuronal circuits. Currently, the cellular and molecular mechanisms that cause or contribute to pathophysiological changes in central nervous system structure and function are not well understood. A number of studies, including ours, have demonstrated a remarkable convergence between structural and functional organization of neuronal circuits and expression of α2δ subunits of voltage gated calcium channels (VGCC). α2δ subunits positively regulate synaptic transmission by increasing plasma membrane expression of VGCC. However, these subunits may also play a pathological role following axonal injury. Expression of α2δ1/2 increases following axonal injury, resulting in aberrant neuron activities associated with chronic pain, spasticity and post-traumatic epilepsy. Whether increased α2δ1/2 expression hijacks the self-repair mechanisms of the central nervous system (CNS) by forcing aberrant plasticity after trauma is not known. Our proposed research seeks to examine whether it is possible to counteract these maladaptive changes by pharmacologically blocking α2δ1/2 in the brain, spinal cord and the retina using a ‘smart’ drug delivery system (SDDS). The SDDS we have demonstrated is made up of polymer-based injectable microspheres that can be manufactured using polymer compositions that fully degrade after drug delivery is complete.

There is a need to develop more effective clinical interventions aimed to improve neurological function and quality of life in individuals afflicted by brain, spinal cord and optic nerve injury and reduce the impact of neurodegenerative diseases, which are a huge economic and emotional burden on society. Currently, there are no effective treatments available to counteract maladaptive changes in the injured brain, spinal cord and eye. Understanding how CNS trauma causes pathophysiological alterations and long-term impairments, including the susceptibility of developing neurodegenerative diseases represents an unmet challenge. Increased α2δ1/2 expression may hijack the self-repair program of the CNS by forcing aberrant plasticity and facilitating synaptic transmission and synaptogenesis after trauma. Given that α2δ1/2 subunits are inhibited by a class of clinically approved drugs called gabapentinoids (e.g., gabapentin and pregabalin), it may pharmacologically block α2δ1/2-mediated maladaptive plasticity by using a polymer based injectable microsphere system for highly localized drug delivery. The use of such a ‘smart’ drug delivery system can allow us to circumvent problems associated with discomfort of multiple daily injections and the unwanted side-effects of dizziness, drowsiness and water retention associated with systemic administration of gabapentinoids.

It is important to understand whether maladaptive plasticity and progressive neurodegeneration that develops following a one-time insult like a head, spinal cord or optic nerve injury may be spatially and temporally controlled for therapeutic gain.

Experimental Methods

Microspheres are typically fabricated from a 3 wt % PCL solution in hexafluoroisopropanol (HFP) flowing through a 14 gauge needle at 1 ml/hour. HFP is chosen due to its rapid evaporation rate that enables the success of electrohydrodynamic processes. A 13 kV potential was applied to the needle containing the polymer solution, triggering the electrospraying process to result in −5 micron diameter particles having a consistently uniform morphology. The electrospray deposition was collected on an aluminum foil platform coated with a 0.5 ml ethanol solution initially containing 12 mg Pluronic F127. The latter greatly improves the dispersability of the as-produced microparticles in aqueous solutions, a factor important in their subsequent passage through a fine glass capillary needle in vivo. Deposition took place over a period of 1 h and resulted in approximately 20 mg of useful microparticles. Gabapentin was incorporated into these particles at 6.7 wt % loading via simple dissolution into the initial polymer solution. Adjustments needed to maintain uniform particle production were made on an as-needed basis.

### Example 3. Injectable, Dispersible Polysulfone-Polysulfone Core-Shell Particles for Optical Oxygen Sensing

Injectable sensors can significantly improve the volume of critical biomedical information emerging from the human body in response to injury or disease. Optical oxygen sensors with rapid response times can be achieved by incorporating oxygen-sensitive luminescent molecules within polymeric matrices with suitably high surface area to volume ratios. Electrospraying utilizes these advances to produce conveniently injectable, oxygen-sensing particles made up of a core-shell polysulfone-polysulfone structure containing a phosphorescent oxygen-sensitive palladium porphyrin species within the core. Particle morphology is highly dependent on solvent identity and electrospraying parameters; DMF was judged to be superior in the creation of uniform, sub-micron particles. Total internal reflection fluorescence (TIRF) microscopy confirmed the existence of both core-shell structure and oxygen sensitivity. The dissolved oxygen response time is rapid (<0.30 s), ideal for continuous real-time monitoring of oxygen concentration. The incorporation of Pluronic F-127 surfactant enables efficient dispersion; selection of an appropriate electrospraying solvent (DMF) yields particles readily injected even through a ˜100 μm diameter needle.

Introduction

In biomedical applications, injection of the sensing platform eliminates the need for more complex surgical implantations that can introduce additional complications and longer recovery times¹⁻³. Luminescent oxygen sensors provide a robust sensor platform that can identify hypoxic areas.⁴ In some cancer treatments, poorly oxygenated areas typically resist traditional chemotherapy and radiation and are associated with an increased likelihood of metastasis⁵⁻⁷ Other potential long-term applications include assessing oxygen levels in ischemic tissue for diabetic patients⁸ and monitoring intrathecal oxygen concentration to assess healing potential after spinal cord injuries. An injectable oxygen sensor with emissions detectable outside the body can provide straightforward monitoring of these conditions.

In general, oxygen-sensitive molecules function based on the dynamic quenching of their luminescent output.¹⁰ Luminescent oxygen sensing offers advantages over traditional methods, such as the Clark electrode, due to their ease of miniaturization and the fact that they do not consume oxygen.¹¹ For these optical oxygen sensors, the emission intensity and phosphorescent lifetime decrease in the presence of oxygen due to dynamic quenching; maximum emission intensity and lifetime occur in the absence of oxygen.¹² When incorporated into a so-called ‘thin’ polymeric film—the most common form—slower response times on the order of many seconds can result.¹² In contrast, rapid response times are achieved when electrospun fibers are the matrix.¹³⁻¹⁵

This work sought to preserve the desirable aspects of electrospun fibers while simultaneously creating a more easily injectable sensor form. Electrospraying was used to incorporate the targeted oxygen-sensitive molecules as shown in FIG. 1. Electrospraying was used to encapsulate these oxygen-sensitive molecules in both micron- and submicron-sized particles.

As an electrohydrodynamic process, electrospraying can be affected by numerous processing parameters: the source-ground distance, the relative core and shell flow rates, polymer concentration, and solvent properties (i.e., vapor pressure).¹⁶ In particular, the magnitude of applied electric field strongly governs behavior.” Unlike electrospinning, successful electrospraying is only achieved within a relatively narrow operational window.^{18,19} Smeets et al. suggested that a successful electrospraying process would only occur when a stable ‘cone-jet mode’ is achieved.¹⁸ Many interacting variables, including polymer chain entanglement, solvent identity, flow rate, and needle tip-to-collector distance, combine to determine the outcome of electrospraying.^{16-18,20,21} Due to the wide variety of conditions that affect particle morphology, achieving precise control over the electrospraying process can be challenging. Many research efforts have examined the relationship between process conditions and the resulting morphology of electrosprayed particles.²²⁻²⁴

However, the bulk of these electrospraying efforts are focused on either traditional single solution electrospraying or coaxial electrospraying. In most cases, coaxial electrospraying is implemented to create an aqueous core.^{16,25} In contrast, Yoon et al. produced polystyrene-polycaprolactone (PS-PCL) and polymethyl methacrylate-polycaprolactone (PMMA-PCL) polymeric core-shell electrosprayed particles.¹⁹ Other notable examples include polyvinylpyrrolidone-shellac²⁶, starch-polydimethylsiloxane²⁷, poly(D,L-lactic-co-glycolic acid)-poly(D,L-lactic acid)²⁸, and poly(L-lactic acid)-poly(D,L-lactic-co-glycolic acid)²⁹ core-shell particles for drug delivery. Solid polymer cores are preferred for luminescent oxygen sensors, because oxygen-sensitive porphyrins and transition metal complexes are prone to agglomeration and self-quenching³⁰, and, therefore, the best performance is achieved for chromophores evenly distributed/dissolved within a solid matrix. It is also desirable that oxygen-sensitive molecules be surrounded by a solid polymer shell to prevent or slow potential leaching into the surrounding biological environment.

Achieving dispersion of electrosprayed particles is another concern for efficient use in biological applications. Solid electrosprayed particles are usually not suitable for injection; instead, they need to be dispersed in a biocompatible medium such as an aqueous solution to acquire ‘injectability.’ However, commonly used biocompatible polymers are often hydrophobic, causing as-electrosprayed particles to aggregate when added to hydrophilic media.³¹ One strategy for preventing agglomeration is the effective hydrolyzation of a particle surface using surfactants.^{31,32} For instance, Seth et al. successfully dispersed surfactant-loaded poly(lactide-co-glycolide) (PLGA) electrosprayed particles in water using bath sonication.³¹

This work created polymer-based, solid core-shell electrosprayed particles that successfully demonstrate the ability to sense dissolved oxygen. Polysulfone (PSU)—chosen due to its toughness, thermal and chemical stability, and biocompatibility^{33,34}—contained the oxygen-sensitive species, palladium (I) meso-tetra(pentafluorophenyl) porphyrin (PdTFPP), while a PSU shell surrounded the PSU+PdTFPP core to prevent leaching of the porphyrin. PSU is a non-resorbable polymeric biomaterial³⁵ with low toxicity and good biocompatibility³⁶⁻³⁸ that has been used in hemodialysis membranes^{36,37} and implantable infusion ports³⁶ Although less is known about the biocompatibility of PdTFPP, no cell toxicity was observed for U251 cells cultured on PdTFPP-containing core-shell electrospun fibers.^{4,15} In addition, the core-shell particle structure was selected to limit the potential for PdTFPP leaching. Although this Pd (II) porphyrin is not currently used in implantable applications to our knowledge, a similar covalently-bound Pd (II) benzoporphyrin derivative is widely used in human implanted oxygen sensors developed by Profusa, Inc.^{39,40} It was first investigated how solvent or solvent mixture properties affected particle morphology, optimizing the morphology as needed by adjusting specific electrospraying parameters. The resulting particles were readily dispersible through the incorporation of a surfactant, Pluronic F-127, along with sonication. Pluronic F-127, also known as Polaxomer 407, exhibits good biocompatibility and low toxicity^{41,42} and is a component in various FDA-approved pharmaceuticals and formulations^{42,43}, as well in LeGoo endovascular occlusion gel.⁴⁴ Injectability was demonstrated by unimpeded particle flow through small, ˜94 μm diameter glass-pulled micropipettes. Lastly, oxygen sensing capabilities were demonstrated via total internal reflection fluorescence (TIRF) microscopy.

Experimental Methods

Materials

PSU (Mₙ˜16,000), tetrahydrofuran (THF), and Pluronic F-127 were acquired from Sigma-Aldrich (St. Louis, MO, USA). Dichloromethane (DCM) was purchased from Fisher Scientific (Waltham, MA, USA). 1,1,1,3,3,3-hexafluoro-2-propanol (HFP) and dimethylformamide (DMF) were obtained from Oakwood Products (West Columbia, SC, USA). PdTFPP was acquired from Frontier Scientific (Logan, UT, USA).

Fabrication of Electrosprayed Core-Shell Particles

The basis for both the ‘core’ and ‘shell’ solution was 1 wt % PSU dissolved in either DCM-HFP blends (50:50, 65:35, and 75:25 by wt.), pure DCM, pure DMF, or pure THF. PdTFPP was added to the core solution at a weight ratio of 1:100 (DMF, THF) or 1:200 (DCM, DCM-HFP) based on polymer weight. The shell solution contained the surfactant Pluronic F-127 at a weight ratio of 1:100. Solutions were stirred on a magnetic stir plate until all solids were visually dissolved.

A coaxial needle (ramé-hart instrument co.; Succasunna, NJ, USA) was used for electrospraying. The core solution traveled through an inner 22-gauge needle, while the shell solution traveled through an outer 14-gauge needle. A 65 mm diameter aluminum dish was generally used as a grounded collector. Based on initial screening tests, various electrospraying parameters (core/shell flow rates, source-to-collector distance, applied voltage) were optimized separately for each solvent. Ultimately, this process led to the selection of the following parameters: 1) DCM-0.1/0.5 mL/hr core-shell flow rates, 20 cm source-to-collector distance, and 20 kV applied voltage 2) 75:25 DCM-HFP-0.3/0.5 mL/hr core-shell flow rates, 20 cm source-to-collector distance, and 20 kV applied voltage 3) DMF-0.3/1 mL/hr core-shell flow rates, 15 cm source-to-collector distance, and 17 kV applied voltage 4) THF-0.3/1 mL/hr core-shell flow rates, 15 cm source-to-collector distance, and 14 kV applied voltage. The dry particles resulting from each of these conditions were transferred to a 17 mm inner diameter glass vial. The vials had previously undergone air plasma treatment for 3 minutes at ˜300 mTorr using a Harrick Plasma cleaner (Ithaca, NY, USA). Then 10 mL of phosphate-buffered saline (PBS) or PBS containing Pluronic F-127 (1 mg/mL) was added. Particle dispersion in PBS was then attempted using a Fisher Scientific FS60 bath sonicator (Waltham, MA, USA) operated for 5 minutes.

Characterization

Scanning Electron Microscopy

For initial assessments of morphology, particles were directly electrosprayed onto aluminum foil (using a net deposition time ≤3 minutes) unless otherwise noted. The foil was then attached atop an aluminum pin stub mount via conductive carbon tape from SPI Supplies (West Chester, PA, USA). All samples were coated with gold prior to imaging. Scanning electron microscopy (SEM) was performed using a FEI SEM (Hillsboro, OR, USA) at 5 kV. ImageJ was used to quantify particle diameter (n=50).

Total Internal Reflection Fluorescence Microscopy

Total internal reflection fluorescence (TIRF) microscopy was performed using a Nikon Eclipse Ti-E inverted microscope (Melville, NY, USA) with 100 mW continuous-wave adjustable power 488 nm laser excitation. Imaging was performed at 100× under oil immersion using Nikon Type A immersion oil (Refractive index at 23° C.: 1.515; Melville, NY, USA). Both fluorescent and differential interference contrast (DIC) images were captured with an Andor iXon3 EMCCD camera at 160 nm/px resolution (Belfast, UK). The particle emission was captured with a Chroma quad-cube filter (Bellows Falls, VT, USA). For TIRF imaging, particles were electrosprayed directly onto a cover slip mounted on a custom flow-through setup. For imaging in aqueous conditions, either water or deoxygenated water was drawn through the setup device. The dissolved oxygen concentration was determined prior to the flow-through experiment using a Hach HQ40d dissolved oxygen meter (Loveland, CO, USA). For response time measurements, water and deoxygenated water alternately flowed into the system while image capture took place. ImageJ was used to quantify particle diameter (n=50).

Results

Many interacting variables affect the success of electrospraying. For instance, a level of polymeric entanglement that is not too high is necessary to allow the transition of falling fiber-based jets into separate droplets.²³ Net polymer entanglement can be most easily controlled via polymer concentration. Ideally, entanglement will be at a level defined as the “semi-dilute moderately entangled regime.”¹⁶ If the polymer concentration is too high, fiber formation becomes favored.¹⁹ If the concentration is too low, the resulting particles may be either collapsed, wrinkled or otherwise deformed.²³ There is generally believed to be an optimal concentration range that allows for dense, spherical particles having minimal porosity while avoiding fiber and/or tailed particle formation.^{19,23,45,46} In this work, trial-and-error yielded an optimal polymer concentration of 1.0 wt % PSU for all reported electrospraying efforts.

In addition, solvent identity has profound effects on particle morphology.^{16,17,46,47} Particle formation involves both solvent evaporation and polymer chain rearrangement.¹⁶ To form the desired rounded morphologies, Wu et al. have stated that the solvent present within the droplets has to evaporate substantially before contact with the collector.^{21,46} However, too-high volatility produces porous electrosprayed particles due to phase separation at the particle surface^{21,46} triggered by surface cooling.⁴⁶ Rapid evaporation can also inhibit polymer chain rearrangement, contributing to the formation of porous, collapsed, or even hollow particles.^{16,20} Therefore, while high vapor pressure has traditionally been seen as an important electrospraying criteria, there are reports of successful electrospraying utilizing low vapor pressure solvents.^{16,48,49} Potential advantages of lower vapor pressure solvents include optimized electrospraying parameters and more ideal solvent/solution properties allowing avoidance of particle collapse due to slower evaporation while also providing more time to achieve spherical particles. For instance, high boiling point solvents can achieve smaller particles with a smoother morphology as slower evaporation allows for sufficient polymer chain rearrangement.^{16,49} Table 1 depicts the vapor pressure and boiling point of various solvents explored in this work.

TABLE 1
Vapor pressure and boiling point for different solvents or
solvent combinations.

| | Vapor Pressure at | Boiling Point |
|---|---|---|
| Solvent | 20° C. [kPa] | [° C.] |
| DCM | 35⁵⁰ | 40 |
| HFP | 14⁵¹ | 59 |
| 75:25 (wt %) DCM:HFP | 32ª | — |
| DMF | 0.52⁵² | 153 |
| THF | 17⁵³ | 66 |

^{a}Estimated value from Raoult's law.

Other relevant solvent parameters include surface tension and viscosity.^{47,54} Solutions that are too viscous can limit the rearrangement of polymer chains during the drying process, which can hinder the formation of smooth, non-porous particles and can possibly lead to deformed particles, beaded fibers, and/or fibers.^{47,54} Surface tension is also highly relevant, because the competition between the force applied by the electric field and the surface tension of the droplet at the needle tip helps to dictate the mode of electrospraying achieved.^{21,55} Additionally, for cone-jet mode electrospraying, droplet size is inversely proportional to surface tension.⁵⁵ Although this work investigated the impact of altering the electrospraying solvent, both the core and shell solvent were identical to avoid potential immiscibility effects.¹⁷

Electrosprayed Particles Using DCM and DCM-HFP

Particle Morphology

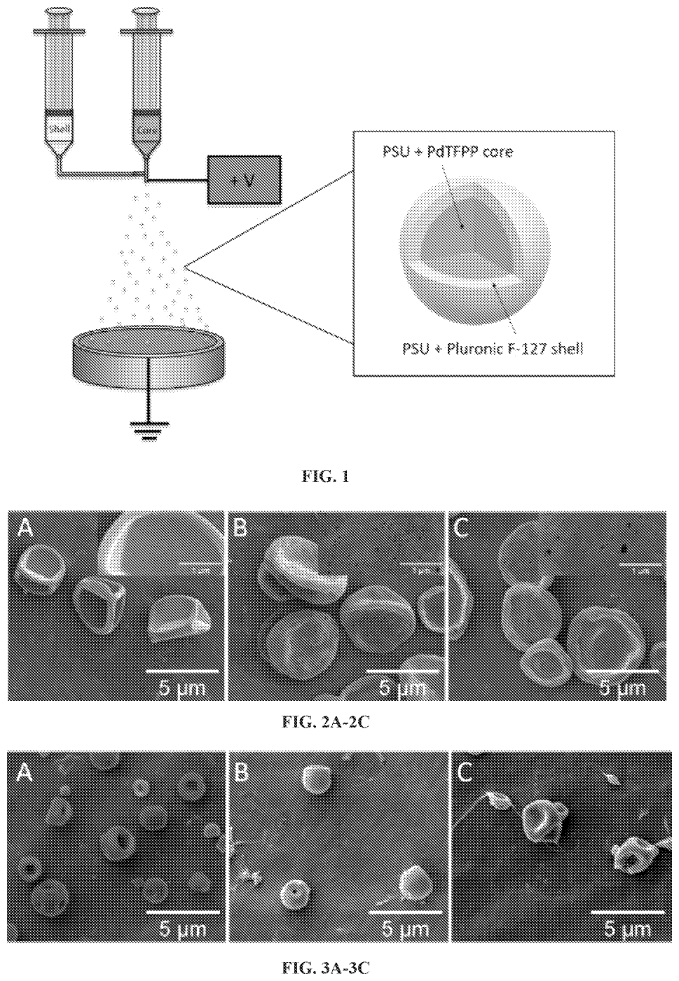

To ensure successful incorporation of oxygen-sensitive porphyrins, they must first exhibit good solubility in the electrospraying solvent. Since PdTFPP is highly soluble in DCM and highly volatile DCM is a common electrospraying solvent^{56,57}, electrosprayed particles were first created using pure DCM as the solvent. Optimal core and shell flow rates were then determined by trial and error. In all cases, there was a noticeable lack of fiber formation and tailed particles. Core/shell flow rates of 0.1/0.5 mL/hr resulted in collapsed particles with interesting geometric shapes (FIG. 2A). However, fine porosity is evident at higher magnifications (FIG. 2A inset). Higher core/shell flow rates of 0.3/0.5 mL/hr and 0.5/0.5 mL/hr yielded significantly more irregular collapsed geometries with larger pores (FIGS. 2B-C). Higher core flow rates may increase the amount of solvent that must escape through the shell, resulting in a net increase in porosity. Although lowering the core flow rate reduced pore size, porosity could not be eliminated. As a solvent, DCM was judged to be less than ideal for this particular application since leaching of the interior porphyrin through the inherent porosity could conceivably occur. Additionally, the highly collapsed nature of these particles was surprising as DCM is a commonly deployed electrospraying solvent¹⁶; however, the core-shell nature of these electrosprayed particles could have been a complicating factor.

As an alternative to highly volatile DCM alone (Table 1), we chose to combine it with the lower vapor pressure and higher boiling point HFP (Table 1). These DCM:HFP blends were investigated to slow overall evaporation while still solubilizing PdTFPP. Although PdTFPP cannot dissolve in pure HFP, it is highly soluble in DCM:HFP blends. In our previous work, we successfully fabricated PdTFPP-containing electrospun PSU fibers using DCM:HFP blends.^{4,15,58}

The addition of HFP during electrospraying successfully eliminated porosity; however, it also appeared to promote fiber formation. When the [HFP] was too high (i.e., 65:35 and 50:50 DCM:HFP), fiber formation became significant (Figure S1 in Supporting Information). Therefore, following these preliminary experiments, we chose 75:25 DCM:HFP as the ideal solvent ratio. Electrospraying parameters were then optimized for this ratio. Core/shell flow rates had a significant impact on particle morphology. Ultimately, 0.3/0.5 mL/hr were selected as the ideal core/shell flow rates for maximizing the formation of spherical particles (FIG. 3B) with this blended solvent system. Core/shell flow rates of 0.1/0.5 mL/hr resulted in concave particles of variable diameters possessing the desired dense surfaces (FIG. 3A). Increasing the core flow rate to 0.3 mL/hr decreased the degree of particle concavity (FIG. 3B), while increasing the flow rate further to 0.5 mL/hr led to the formation of increased particle non-uniformity and fiber formation (FIG. 3C). Faramarzi et al. encountered similar trends for electrosprayed PLGA, whereby increased flow rate aided the transition from collapsed to spherical PLGA particles. Too high of flow rates led to deformed, non-uniform particles, accompanied by ‘tailed’ particles and fibers.²³ For coaxial electrospraying in general, the shell flow rate should be no slower than the core flow rate or the shell may not fully encapsulate the core solution within the Taylor cone at the needle tip.¹⁹ Although the effect of flow rate is poorly documented for polymeric core-shell particles, Yoon et al. also observed that the shell flow rate should be larger than that of the core for improved uniformity and sphericity.¹⁹

In some electrospraying systems, it can be challenging to avoid collapsed particles^{46,49} as the rapid solvent evaporation can disrupt particle sphericity.⁵⁹ For instance, Wu et al. fabricated collapsed polycaprolactone (PCL) electrosprayed particles using a mixed solvent, chloroform-acetone, and attributed this shape to polymer- and solvent-rich phases that formed the particle wall and the hollowed-out region, respectively.⁴⁶ The highly volatile nature of DCM (Table 1) likely caused rapid evaporation from the droplet surface, creating a structurally weak ‘crust’^{20,22}, inevitably resulting in collapsed particles regardless of selected core/shell flow rates (FIG. 2). Collapsed particles still occurred in some instances with DCM-HFP, but improved sphericity (FIG. 3B) was achievable with careful control over core/shell flow rates in this less volatile solvent blend (Table 1).

Core-Shell Structure and Oxygen Sensing Capabilities

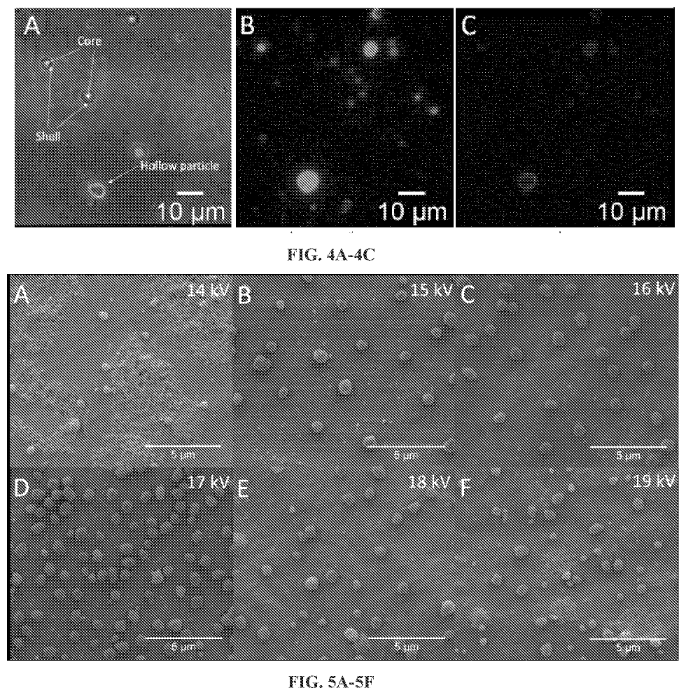

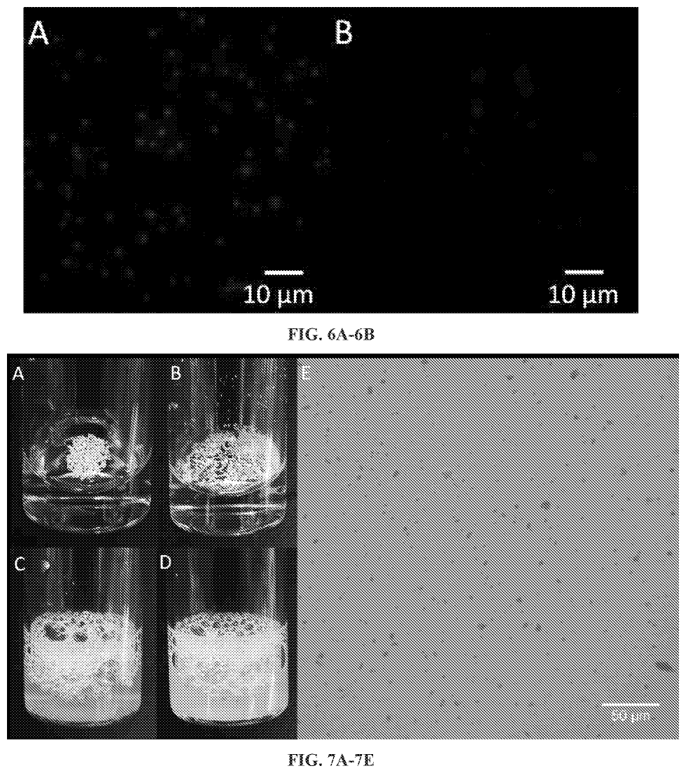

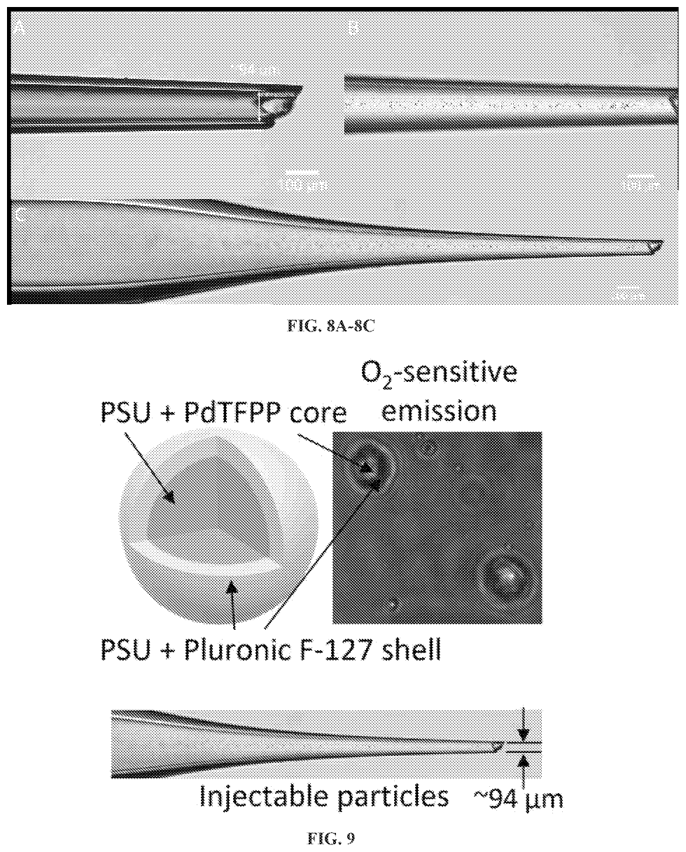

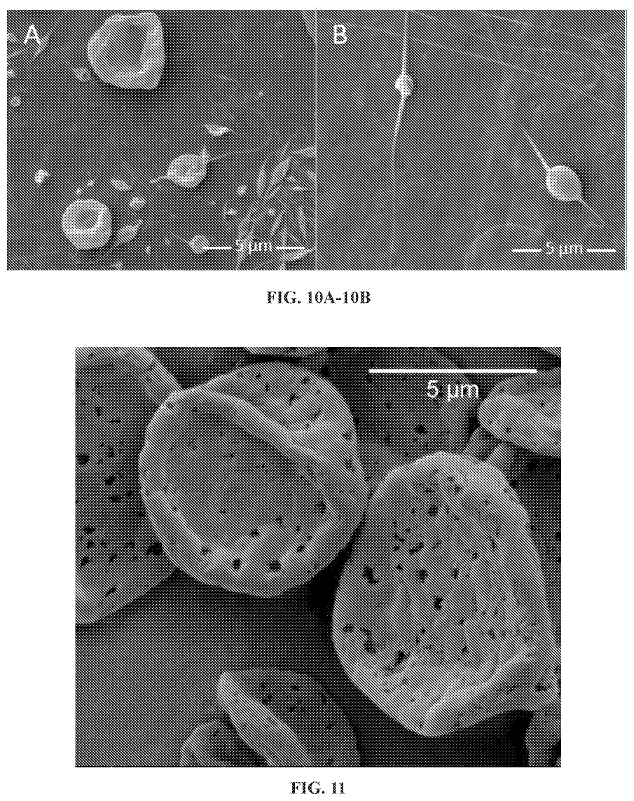

Following successful particle formation, FIG. 4A provides TIRF data that demonstrates the core-shell structure of PSU (PdTFPP)-PSU (Pluronic F-127) particles fabricated using a mixed DCM-HFP solvent. In this merged fluorescent/DIC image, the non-luminescent Pluronic F-127-containing shell is visible as a dark ring on the outside of each electrosprayed particle. Meanwhile, the phosphorescent core containing PdTFPP is evident within the dark shell. However, some of the larger electrosprayed particles appear hollow as indicated by the presence of a dark shell, a concentric phosphorescent ‘core’ and a non-luminescent interior. There exists a fairly wide variation in particle size with an average diameter of 2.12±1.47 μm; the presence of large, hollow particles may contribute to this non-uniformity.

Solid, spherical particles (i.e., those shown in FIG. 3B) could be used as oxygen-sensitive microspheres. The solid PSU core is an optimal matrix to contain oxygen-sensitive species and prevent self-quenching. Meanwhile, the outer shell structure (FIG. 4A) should prevent leaching of the internally contained oxygen-sensitive species. FIGS. 4B-C demonstrate the dissolved oxygen sensing capability of PSU (PdTFPP)-PSU (Pluronic F-127) particles formed using DCM-HFP. The phosphorescent output of the particles is low in untreated water at typical dissolved oxygen levels (FIG. 4C) but increases significantly in deoxygenated water (FIG. 4B), since the phosphorescence output is quenched in the presence of oxygen.

Measured response and recovery times for dissolved oxygen sensors can often be dominated by the relatively slower processes needed to alter the dissolved oxygen concentration; however, the custom TIRF flow-through setup used in this work allowed for rapid exchange of dissolved oxygen with the surrounding solution. The electrosprayed particles produced in this work possess a rapid response time as demonstrated during the switch from normoxic (dissolved oxygen content: 8.7 mg/L) to deoxygenated water (dissolved oxygen content: 0.23 mg/L) which required only 0.29±0.02 s (n=4). Meanwhile, the change from deoxygenated to normoxic water required only 0.13±0.06 s (n=4). A response time (deoxygenated to oxygenated conditions) shorter than the recovery time (oxygenated to deoxygenated conditions) has been previously observed for other luminescent dissolved oxygen sensors.^{14,15,60-62} This difference is likely the result of the matrices (e.g., polysulfone) exhibiting enhanced permeability and diffusivity for oxygen versus nitrogen.^{63,64} Also, note that the measured response and recovery times are significantly faster than those commonly observed for other dissolved oxygen sensors. The rapid response is likely a result of the small diffusion distances and high surface area of the electrosprayed particles. Given that the measured response and recovery times are likely significantly faster than most biologically-driven changes in oxygen concentration, these electrosprayed particles would be ideal for use in continuous real-time monitoring of dissolved oxygen in vivo. This performance demonstrates that PdTFPP-bearing electrosprayed core-shell particles show great promise for biomedical applications. For instance, the DCM-HFP particles are large and bright enough to be monitored individually. This characteristic could be useful to examine oxygen concentration gradients during in vitro fluorescence microscopy experiments. However, greater particle uniformity and smaller particle size are desired for truly injectable in vito applications. Therefore, we then sought an alternative solvent to form smaller yet uniformly sized particles for increased injectability. When exploring alternative solvent systems, we also sought to focus on single solvent systems to avoid the potential for non-uniformities that could be caused by any miscibility issues or by preferential evaporation of the lower boiling point solvent first.⁶⁵

Electrosprayed Particles Using DMF

Particle Morphology

As mentioned previously, the volatility of solvents has a profound impact on particle morphology. Table 1 suggested that a solvent with lower volatility/vapor pressure than DCM or DCM-HFP may be useful. Tetrahydrofuran (THF) (Table 1) was examined briefly, but the resulting particles were both collapsed and extremely porous (see Figure S2 in Supporting Information) despite this lower vapor pressure. Surprisingly, considering its remarkably low vapor pressure (Table 1), DMF is commonly employed for electrohydrodynamic processes due to its superior ability to create spherical morphologies^{16,48,49} if solution properties and electrospraying parameters can be optimized to avoid particle collapse and achieve spherical particles. The slower evaporation allows time for the polymer chains to rearrange within the droplets and form smaller but structurally ‘stronger’ particles.^{16,20,52} It was established that DMF, fortunately, was an excellent solvent for PdTFPP.

The deployment of DMF as the solvent for both shell and core solutions successfully resulted in dense, spherical particles (FIG. 5). After initial screening tests, optimal core and shell flow rates were determined to be 0.3 and 1 mL/hr, respectively. As was noted for DCM-HFP particles, a higher shell flow rate is critical for efficient encapsulation of the core. The applied voltage was found to have significant effects on the electrospraying process; particles electrosprayed at several voltages are shown in FIG. 5. The operational window for successful drip-free electrospraying was 17-18 kV (FIGS. 5D-E). At these applied voltages, a stable ‘cone-jet’ mode^{18,19} and optimal particle yield efficiency were achieved. In this regime, discrete particles having relatively uniform dimensions (0.74±0.11 μm for particles electrosprayed at 17 kV) appeared. If the voltage was too low (i.e., 14 kV, FIG. 5A), both solid particles and occasional dripping were observed¹⁸. In contrast, ‘multi-jet’ mode^{18,19} was encountered when the voltage was too high (i.e., 19 kV, FIG. 5F), leading to the production of inconsistent particle dimensions.

Compared to particles created using DCM and DCM-HFP (FIGS. 2 and 3), electrosprayed PSU-PSU particles using DMF demonstrated improved particle morphologies that are preferable for injectable biosensors and were downselected for the remainder of the work. Note that DMF has a higher boiling point and a lower vapor pressure (Table 1) than other commonly used electrospraying solvents,¹⁶ demonstrating that high vapor pressure solvents are not always necessary for successful electrospraying.

Oxygen Sensing Capabilities

FIG. 6 provides TIRF data demonstrating the dissolved oxygen sensing capabilities of the DMF-based PSU (PdTFPP)-PSU (Pluronic F-127) particles. As expected, the phosphorescent signal of the particles is clearly visible in deoxygenated water (FIG. 6A) but is substantially lower in normoxic water given the much higher dissolved oxygen concentration which strongly quenches PdTFPP's phosphorescence (FIG. 6B). Since the diameters of these sub-micron DMF-based particles (0.74±0.11 μm) are similar to visible light wavelengths and the shell thicknesses are even smaller, distinctive core-shell structures (FIG. 4A) could not be resolved using TIRF.

Particle Dispersion and Injectability

Particles electrosprayed using DMF at 17 kV were initially poorly dispersible in PBS (FIG. 7A). The lack of initial dispersion is unsurprising since hydrophobic electrosprayed particles are known to aggregate in water.^{31,66} While salt-based solutions have been used to electrostatically stabilize far smaller (20-100 nm) colloidally-based suspensions, e for PBS at 25° C. is only 79.0⁶⁷, far smaller than values (usually >100⁶⁸) typically used to achieve electrostatic stabilization at non-neutral pH's. Although it is possible that the salt content and pH of PBS could have some benefits in promoting dispersion, the use of PBS buffer (versus water) alone was not sufficient to achieve particle dispersion. Aggregation is a major challenge hindering the use of electrosprayed particles as injectable biosensors since large enough aggregates could easily clog needles. However, many widely used biocompatible polymers—including PCL, polylactic acid (PLA), polyglycolic acid (PGA) and PLGA—are initially hydrophobic and, therefore, their electrosprayed particles initially aggregate in the aqueous condition, making them unsuitable for injection as biosensors.³¹ In many instances, surfactant incorporation has been shown to discourage agglomeration as a result of increased surface charge.^{31,32,45} In addition, surfactant molecules blended in electrospraying or electrospinning solutions have been reported to preferentially diffuse to and present on the particle or fiber surface.^{31,69} For electrospun poly(lactide-co-glycolide) (PLGA) blended with Pluronic F-108, Vasita et al. speculated that unlike the hydrophobic poly(propylene glycol) (PPG) blocks, the hydrophilic poly(ethylene glycol) (PEG) blocks of the surfactant may not integrate efficiently with the hydrophobic polymer chains and, therefore, may be projected toward the fiber surface.⁶⁹ The presence of PEG blocks on the surface can enhance the wettability of hydrophobic polymers, allowing for improved dispersion in water in the case of electrosprayed particles.^{31,69}

However, the initial particle suspension surprisingly resisted a five-minute sonication treatment (FIG. 7B) despite the presence of Pluronic F-127 surfactant in the shell. Significantly improved particle dispersion was achieved when Pluronic F-127 was directly introduced to the PBS solution (1 mg/ml) to create an adsorbed surfactant coating for enhanced surface wettability. In FIG. 7C, the initially transparent PBS solution became cloudy immediately after adding Pluronic F-127, indicating that PSU-PSU particles started to disperse prior to sonication. In FIG. 7D, five minutes of sonication appeared to sufficiently disperse almost all aggregates, forming a relatively uniform particle suspension and readying them for direct injection. Particles remained dispersed for several days post-sonication. A few droplets of the suspension were added to a glass slide and examined under optical microscopy to confirm the high degree of dispersion that remained four days post-sonication (FIG. 7E).

FIG. 8 demonstrates the injectability of a typical PSU-PSU particle suspension using a standard, glass-pulled micropipette employed in brain, spinal cord and eye research.^{70,71} The opening of this particular glass-pulled micropipette is approximately 94 μm (FIG. 8A), in the range of 33-34 gauge needles used in microfluidics research. In spite of its small size, the needle inner diameter is significantly larger than discrete DMF-produced particles or even the small clusters visible in FIG. 7E. Therefore, these particle-bearing suspensions exhibited unimpeded flow through these micropipettes (FIGS. 8B-C).

Conclusion

We successfully established that electrospraying can be used to create solid, injectable core-shell particles that can function as useful oxygen sensors. In contrast to more commonly used liquid cores for drug release applications, the solid polysulfone core allowed for incorporation of a phosphorescent oxygen-sensitive porphyrin in a manner that prevents self-quenching. The dissolved oxygen response time of <0.30 s is consistent with past measurements from electrospun fibers and indicates that these particles show great potential for use as injectable, real-time optical oxygen sensors capable of rapidly adapting to small changes in localized oxygen levels. Thanks to careful control over electrospraying parameters, injectable, oxygen-sensing polymeric core-shell electrosprayed particles were fabricated. Use of DMF or DCM-HFP as the electrospraying solvent produced demonstratively non-porous particles avoiding the highly collapsed and porous particles associated with the use of pure DCM. However, the decreased particle size and increased uniformity of DMF-based particles (0.74±0.11 μm) are preferable in injectable applications; therefore, the potential utility of DMF-based particles as injectable was investigated, optical oxygen sensors. Particle dispersion achieved via sonication and incorporation of a surfactant enabled successful demonstrations of injectability through needles 5-8 times smaller than those routinely used in human medicine. Based on these achievements, we can envision numerous potential future applications, including the examination of oxygen gradients in the neighborhood of a brain (traumatic or ischemic stroke) or spinal cord injury, in the retina following optic nerve injury or glaucoma, or localized subcutaneous quantification of variable in vivo oxygen concentrations associated with restrictions to blood flow to the extremities caused by diabetes.

### REFERENCES

1. Pichorim, S. F.; Abatti, P. J. IEEE Trans. Biomed. Eng. 2006, 53, 921.
2. Johannessen, E.; Krushinitskaya, O.; Sokolov, A.; Philipp, H.; Hoogerwerf, A.; Hinderling, C.; Kautio, K.; Lenkkeri, J.; Strömmer, E.; Kondratyev, V.; Tønnessen, T. I.; Mollnes, T. E.; Jakobsen, H.; Zimmer, E.; Akselsen, B. J. diabetes Sci. Technol. 2010, 4, 882.
3. Marsh, J. R.; Gates, R. S.; Day, G. B.; Aiken, G. E.; Wilkerson, E. G. 2008, 0300, 1.
4. Xue, R.; Nelson, M. T.; Teixeira, S. A.; Viapiano, M. S.; Lannutti, J. J. Biomaterials 2016, 76, 208.
5. Gatenby, R. A.; Kessler, H. B.; Rosenblum, J. S.; Coia, L. R.; Moldofsky, P. J.; Hartz, W. H.; Broder, G. J. Int. J. Radiat. Oncol. Biol. Phys. 1988, 14, 831.
6. Shannon, A. M.; Bouchier-Hayes, D. J.; Condron, C. M.; Toomey, D. Cancer Treat. Rev. 2003, 29, 297.
7. Brizel, D. M.; Scully, S. P.; Harrelson, J. M.; Brizel, M.; Harrelson, M.; Layfleld, J.; Bean, M.; Prosnitz, R.; Dewhirst, M. W. Cancer Res. 1996, 56, 941.
8. Faglia, E.; Clerici, G.; Caminiti, M.; Quarantiello, A.; Curci, V; Morabito, A. Eur. J. Vasc. Endovasc. Sug. 2007, 33, 731.
9. Verma, K.; Mortazavi, M. M.; Griessenauer, C. J.; Adeeb, N.; Harmon, O. A.; Tubbs, R. S.; Theodore, N. Clin. Anat. 2014, 28, 27.
10. Amao, Y. Microchim. Acta 2003, 143, 1.
11. Wang, X.-D.; Chen, H.-X.; Zhao, Y.; Chen, X., Wang, X. Trends Anal. Chem. 2010, 29, 319.
12. Okura, I. Photosensitization of Porphyrins and Phthalocyanines; 1st ed.; Kodansha: Tokyo, 2000.
13. Xue, R.; Behera, P.; Viapiano, M. S.; Lannutti, J. J. Mater Sci. Eng. C 2013, 33, 3450.
14. Xue, R.; Behera, P.; Xu, J.; Viapiano, M. S.; Lannutti, J. J. Sensors Actuators, B Chem. 2014, 192, 697.
15. Xue, R.; Ge, C.; Richardson, K.; Palmer, A.; Viapiano, M.; Lannutti, J. J. ACS Appl. Mater. Interfaces 2015, 7, 8606.
16. Bock, N.; Dargaville, T. R.; Woodruff, M. A. Prog. Polym. Sci. 2012, 37, 1510.
17. Chakraborty, S.; Liao, I. C.; Adler, A.; Leong, K. W. Adv. Drug Deliv Rev 2009, 61, 1043.
18. Smeets, A.; Clasen, C.; Van den Mooter, G. Eur. J. Pharm. Biopharm. 2017, 119, 114.
19. Yoon, K. H.; Unyong, J.; Eun, C. C. Langmuir 2008, 24, 2446.
20. Yao, J.; Kuang Lim, L.; Xie, J.; Hua, J.; Wang, C. H. J. Aerosol Sci. 2008, 39, 987.
21. Wu, Y.; Clark, R. L. J. Biomater. Sci. Polym. Ed. 2008, 19, 573.
22. Huang, X.; Gao, J.; Li, W.; Xue, H.; Li, R. K. Y.; Mai, Y. Mater. Des. 2017, 117, 298.
23. Faramarzi, A.-R.; Barzin, J.; Mobedi, H. Fibers Polym. 2016, 17, 1806.
24. Almeria, B., Deng, W.; Fahmy, T. M.; Gomez, A. J. Colloid Interface Sci. 2010, 343, 125.
25. Xie, J.; Ng, W. J.; Lee, L. Y; Wang, C. H. J. Colloid Interface Sci. 2008, 317, 469.
26. Wang, K.; Wen, H.; Yu, D.; Yang, Y.; Zhang, D. Mater. Des. 2018, 143, 248.
27. Pareta, R.; Edirisinghe, M. J. J. R. Soc. Interface 2006, 3, 573.
28. Xu, Q.; Qin, H.; Yin, Z.; Hua, J.; Pack, D. W.; Wang, C.-H. Chem. Eng. Sci. 2013, 104, 330.
29. Nie, H.; Dong, Z.; Arifin, D. Y; Hu, Y.; Wang, C. H. J. Biomed. Mater. Res.—Part A 2010, 95A, 709.
30. Grenoble, S.; Gouterman, M.; Khalil, G.; Callis, J.; Dalton, L. J. Lumin. 2005, 113, 33.
31. Seth, A.; Katti, D. S. Int. J. Nanomedicine 2012, 7, 5129.
32. Santander-Ortega, M. J.; Jodar-Reyes, A. B.; Csaba, N.; Bastos-Gonzalez, D.; Ortega-Vinuesa, J. L. J. Colloid Interface Sci. 2006, 302, 522.
33. Hergenrother, P. M. High Perform. Polym. 2003, 15, 3.
34. Yilmaz, G.; Toiserkani, H.; Demirkol, D. O.; Sakarya, S.; Timur, S.; Torun, L.; Yagci, Y. Mater. Sci. Eng. C 2011, 31, 1091.
35. Biomaterials Science: An Introduction to Materials in Medicine; Ratner, Buddy D.; Hoffman, Allan S.; Schoen, Frederick J.; Lemons, J. E., Ed.; 2nd editio.; Elsevier Academic Press: San Diego, 2004.
36. El-Hibri, M. J.; Axelrad, S. W. In Handbook of Thermoplastics; Olabisi, O.; Adewale, K., Eds.; CRC Press: Boca Raton, 2016; pp 419.
37. Wenten, I. G.; Aryanti, P. T. P.; Hakim, A. N.; Himma, N. F. J. Membr. Sci. Res. 2016, 2, 78.
38. Pena, B.; Gumi, T. Curr. Org. Chem. 2013, 17, 22.
39. Montero-Baker, M. F.; Au-Yeung, K. Y.; Wisniewski, N. A.; Gamsey, S.; Morelli-Alvarez, L.; Mills, J. L.; Campos, M.; Helton, K. L. J. Vasc. Surg. 2015, 61, 1501.
40. Kanick, S. C.; Schneider, P. A.; Klitzman, B.; Wisniewski, N. A.; Rebrin, K. Microvasc. Res. 2019, 124, 6.
41. Ricci, E. J.; Lunardi, L. O.; Nanclares, D. M. A.; Marchetti, J. M. Int. J. Pharm. 2005, 288, 235.
42. Giuliano, E.; Paolino, D.; Fresta, M.; Cosco, D. Pharmaceutics 2018, 10, 159.
43. Dumortier, G.; Grossiord, J. L.; Agnely, F.; Chaumeil, J. C. Pharm. Res. 2006, 23, 2709.
44. FDA U.S. Food & Drug Administration: Premarket Approval (PMA). https://www.accessdata.fda.gov/scripts/cdrh/cfdocs/cfpma/pma.cfm?ID=P110003 (accessed Dec. 31, 2020).
45. Xie, J.; Lim, L. K.; Phua, Y.; Hua, J., Wang, C. H. J. Colloid Interface Sci. 2006, 302, 103.
46. Wu, Y Q.; Clark, R. L. J. Colloid Interface Sci. 2007, 310, 529.
47. Niu, B.; Shao, P.; Luo, Y.; Sun, P. Food Hydrocoll. 2020, 99.
48. Hao, X.; Lu, X.; Li, Z.; Zhao, Y; Shang, T.; Yang, Q.; Wang, C.; Li, L. J. Appl. Polym. Sci. 2006, 102, 2889.
49. Park, C. H.; Lee, J. J. Appl. Polym. Sci. 2009, 114, 430.
50. ThermoFisher Scientific DCM MSDS 2019.
51. ThermoFisher Scientific HFP MSDS 2018.
52. Wu, X. F.; Zhou, Z.; Zholobko, O.; Jenniges, J. J.; Baatz, B.; Ahmadi, M.; Chen, J. J. Appl. Phys. 2020, 127.
53. Piñeiro, Á.; Brocos, P.; Amigo, A.; Pintos, M.; Bravo, R. J. Chem. Thermodyn. 1999, 31, 931.
54. Boda, S. K.; Li, X.; Xie, J. J. Aerosol Sci. 2019, 125, 164.
55. Jaworek, A.; Sobczyk, A. T. J. Electrostat. 2008, 66, 197.
56. Prabhakaran, M. P.; Zamani, M.; Felice, B.; Ramakrishna, S. Mater. Sci. Eng. C 2015, 56, 66.
57. Wu, Y.; Kennedy, S. J.; Clark, R. L. J. Biomed. Mater. Res. Part B Appl. Biomater. 2008, 90, 381.
58. Presley, K. F.; Stang, M. A.; Cheong, S.; Marjo, C. E.; Spiegler, E. N.; Lannutti, J. J. Sensors Actuators B. Chem. 2019, 283, 269.
59. Fantini, D.; Zanetti, M.; Costa, L. Macromol. Rapid Commun. 2006, 27, 2038.
60. Tian, Y; Shumway, B. R.; Meldrum, D. R. Chem. Mater. 2010, 22, 2069.
61. Tian, Y.; Shumway, B. R., Gao, W.; Youngbull, C.; Holl, M. R.; Johnson, R. H.; Meldrum, D. R. Sensors Actuators, B Chem. 2010, 150, 579.
62. Presley, K.; Hwang, J.; Cheong, S.; Tilley, R.; Collins, J.; Viapiano, M.; Lannutti, J. Mater. Sci. Eng. C 2017, 70, 76.
63. Pixton, M. R.; Paul, D. R. Polymer (Guildf). 1995, 36, 3165.
64. Haraya, K.; Hwang, S. T. J. Memb. Sci. 1992, 71, 13.
65. Yoshida, M.; Miyashita, H. Chem. Eng. J. 2002, 86, 193.
66. Almeria, B.; Gomez, A. J. Colloid Interface Sci. 2014, 417, 121.
67. Chan, V; McKenzie, S. E.; Surrey, S.; Fortina, P.; Graves, D. J. J. Colloid Interface Sci. 1998, 203, 197.
68. Carrique, F.; Arroyo, F. J.: Jimenez, M. L.: Delgado, A. V. J. Chem. Phys. 2003, 118, 1945.
69. Vasita, R.; Mani, G.; Agrawal, C. M.; Katti, D. S. Polymer (Guildf). 2010, 51, 3706.
70. Sun, W.; Larson, M. J. E.; Kiyoshi, C. M.; Annett, A. J.; Stalker, W. A.; Peng, J.; Tedeschi, A. J. Clin. Invest. 2020, 130, 345.
71. Tedeschi, A.; Dupraz, S.; Laskowski, C. J.; Xue, J.; Ulas, T.; Beyer, M.; Schultze, J. L.; Bradke, F. Neuron 2016, 92, 419.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A polymeric microparticle comprising:
a polymeric core comprising a first polymer and an active agent susceptible to abuse; and a polymeric shell comprising a second polymer;
wherein the first and second polymers are biocompatible polymers; and
wherein at least one of the first polymer or the second polymer is a non-erodible biocompatible polymer.

2. The polymeric microparticle of claim 1, wherein the polymeric shell further comprises a dispersing agent.

3. The polymeric microparticle of claim 1, wherein the active agent susceptible to abuse comprises a gabapentinoid.

4. The polymeric microparticle of claim 3, wherein the gabapentinoid comprises gabapentin or pregabalin, or a pharmaceutically acceptable salt thereof.

5. The polymeric microparticle of claim 1, wherein the biocompatible polymer comprises collagen, poly (alpha esters such as poly (lactate acid), poly (glycolic acid), polyorthoesters and polyanhydrides and their copolymers, polyglycolic acid and polyglactin, cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polysulfide, polysulfone, polycaprolactone, polytetrafluoroethylene, polythioether, polytriazole, polyurethane, polyvinyl, polyvinylidene fluoride, or copolymers and blends thereof

6. The polymeric microparticle of claim 1, wherein the biocompatible polymer comprises a polysulfone, polycaprolactone, or any combination thereof.

7. The polymeric microparticle of claim 1, wherein the non-erodible biocompatible polymer comprises polysulfone, poly(ethylene-co-vinyl acetate) (EVA), polyvinylalcohol,

8. (canceled)

9. The polymeric microparticle of claim 1, wherein the dispersing agent comprises polyethylene glycol, poloxomer, or a combination thereof.

10. The polymeric microparticle of claim 9, wherein the dispersing agent comprises a poloxomer.

11. The polymeric microparticle of claim 9, wherein the poloxomer is defined by:
(PEO)ₓ(PPO)_{y}(PEO)ₓ,
wherein
PEO is poly(ethylene oxide),
PPO is poly(propylene oxide),
x can each be an integer from 2 to 130, and
y can be an interfere from 15 to 67.

12. The polymeric microparticle of claim 9, wherein the poloxamer comprises poloxamer 407.

13. (canceled)

14. The polymeric microparticle of claim 1, wherein the active agent susceptible to abuse is present in a weight loading of from 0.1 wt. % to 50 wt. % in the polymeric microparticle

15. The polymeric microparticle of claim 2, wherein the dispersing agent is present in an amount of from 0.01 wt. % to 10 wt. % in the polymeric microparticle.

16. The polymeric microparticle of claim 1, wherein the microparticles exhibits sustained, zero-ordered release the active agent susceptible to abuse over a period of days to weeks.

17. The polymeric microparticle of claim 1, wherein the microparticles releases the drug over a period of days to weeks.

18. A pharmaceutical composition for localized drug delivery, the composition comprising a population of polymeric microparticles of claim 1 dispersed within a pharmaceutically acceptable carrier.

19. An abuse-resistant pharmaceutical composition, the composition comprising a population of polymeric microparticles of claim 1 dispersed within a pharmaceutically acceptable carrier.

20. The composition of claim 18, wherein the pharmaceutically acceptable carrier comprises a dispersing agent.

21-26. (canceled)

27. A method of the treating a neurodegenerative disorder in a subject in need thereof, comprising administering an effective amount of the polymeric microparticles of claim 1.

28-30. (canceled)

31. A method of preparing the polymeric microparticles of claim 1 comprising:
(a) dissolving or dispersing the first polymer in an organic solvent to generate a first polymer solution/dispersion;
(b) dissolving or dispersing the second polymer in an organic solvent to generate a shell solution;
(c) adding the active agent susceptible to abuse to the first polymer solution/dispersion of step (a) to generate a core solution;
(d) electrospraying the core solution and the shell solution onto a pre-treated dish; and
(e) collecting the polymeric microparticles on the pre-treated dish.

32-50. (canceled)
